(19) United States
(12) Patent Application Publication
(10) Pub. No.: US 2026/0000280 A1
(43) Pub. Date: Jan. 1, 2026

(54) MEDICAL DEVICE ACTUATION COMPONENTS, ASSEMBLIES, AND ASSOCIATED METHODS

(71) Applicant: Boston Scientific Scimed, Inc., Maple Grove, MN (US)

(72) Inventors: Kirsten VIERING, Newton, MA (US); William J. ABNER, Rutland, MA (US)

(73) Assignee: Boston Scientific Scimed, Inc., Maple Grove, MN (US)

(21) Appl. No.: 19/252,185

(22) Filed: Jun. 27, 2025

Related U.S. Application Data
(60) Provisional application No. 63/665,600, filed on Jun. 28, 2024.

(51) Int. Cl. A61B 1/00; A61B 1/005

(52) U.S. Cl. CPC A61B 1/00158; A61B 1/00006; A61B 1/00016; A61B 1/00032; A61B 1/00114; A61B 1/0052

(57) ABSTRACT

A medical system may include an actuator assembly. The actuator assembly may include an actuator housing. The actuator housing may include a first side and a second side opposite the first side, an electrical component, an actuator disposed on the first side, exposed to a user of the medical system, and operably coupled to the electrical component, and an actuator coupler proximate to the second side. The medical system may further include a medical device. The medical device may include a handle. The handle may include a handle housing, an umbilicus for electrically connecting the handle to an electrical controller, and a receiver assembly disposed on the handle housing. The receiver assembly may include a receiver coupler. The actuator coupler may be configured to removably couple to the receiver coupler.

### CROSS-REFERENCE TO RELATED APPLICATIONS

This application claims the benefit of priority under 35 U.S.C. § 119 from U.S. Provisional Application No. 63/665,600, filed on Jun. 28, 2024, which is incorporated by reference herein in its entirety.

### TECHNICAL FIELD

Various aspects of this disclosure relate generally to medical device actuation components, assemblies, and associated methods. In particular, aspects of this disclosure pertain to various configurations of actuators for a handle of a medical device, among other aspects.

### BACKGROUND

Medical scope devices, such as endoscopes, may include a handle and a shaft, and the shaft may be insertable into a body lumen of a subject. The handle may include one or more actuators, including buttons, knobs, levers, etc., to control aspects of the medical device such as image capture, suction, irrigation, light control, accessory control, and articulation. Therefore, a need exists for systems, devices, and methods for actuators for medical devices.

### SUMMARY

The disclosure includes systems, devices, and methods for reusable medical device buttons to, for example, use a same button assembly across multiple single use medical devices. Each of the aspects disclosed herein may include one or more of the features described in connection with any of the other disclosed aspects.

A medical system may include an actuator assembly. The actuator assembly may include an actuator housing. The actuator housing may include a first side and a second side opposite the first side, an electrical component, an actuator disposed on the first side, exposed to a user of the medical system, and operably coupled to the electrical component, and an actuator coupler proximate to the second side. The medical system may further include a medical device. The medical device may include a handle. The handle may include a handle housing, an umbilicus for electrically connecting the handle to an electrical controller, and a receiver assembly disposed on the handle housing. The receiver assembly may include a receiver coupler. The actuator coupler may be configured to removably couple to the receiver coupler.

Any of the systems, devices, and methods disclosed herein may include any of the following features. The electrical component may include a battery, a transmitter or transceiver, and a processor. The battery, the transceiver, and the processor may be electrically connected. The processor may be configured to detect actuation of the actuator. The transmitter or transceiver may be configured to transmit a signal indicating the actuation to the electrical controller. The receiver assembly may be disposed on a front side of the handle housing. The umbilicus may be coupled to a protrusion extending from a left side of the handle housing or a front side of the handle housing. The medical system may further include a control knob and a locking mechanism operably coupled to the control knob. The control knob and the locking mechanism may be disposed on a right side of the handle housing. The actuator coupler may include an actuator magnet. The receiver coupler may include a receiver magnet. The receiver coupler may include a first coupler arm and a second coupler arm, a first side wall joined to the first coupler arm, and a second side wall joined to the second coupler arm, and an inner wall joined to the first side wall and the second side wall. The first coupler arm and the second coupler arm may be configured to snap-fit onto the actuator assembly. The actuator assembly may include a flexible portion and a rigid portion. The rigid portion may include the actuator. The electrical component may further include an actuator electrical connector. The receiver assembly may further include a receiver electrical connector. Actuation of the actuator may correspond with transmission of a signal from the actuator electrical connector to the receiver electrical connector. The receiver electrical connector may be electrically connected to the umbilicus. The signal may be transmitted wirelessly. The actuator assembly and the receiver assembly may be correspondingly shaped. The receiver assembly may include a recess. The recess may be configured to inhibit lateral movement of the actuator housing relative to the receiver assembly. The receiver coupler may include a strap. The electrical controller may be configured to control, based on the signal, at least one of an image capture function or a light control function of the medical device.

A medical system may include an actuator assembly. The actuator assembly may include a first side having an actuator, a second side having a first magnet, and an electrical component. The medical system may further include a medical device handle including a receiver. The receiver may include a second magnet configured to interact with the first magnet in order to removably couple the actuator assembly to the receiver.

Any of the systems, devices, and methods disclosed herein may include any of the following features. The electrical component may include a battery, a transmitter or transceiver, and a processor. The battery, the transceiver, and the processor may be electrically connected. The transmitter or transceiver may be configured to transmit a signal to an electrical controller indicating that the actuator has been actuated. The electrical controller may be configured to control, based on the indication, at least one of an image capture function or a light control function of a distal tip of a shaft to the medical device handle.

A medical system may include an actuator assembly. The actuator assembly may include a transmitter or transceiver, an actuator operably coupled to transmitter or transceiver, and a first coupler. The medical system may further include a medical device. The medical device may include a handle, an umbilicus for electrically connecting the handle to an electrical controller, and a receiver assembly disposed on the handle. The receiver assembly may include a second coupler and a recess for removably receiving the actuator assembly. The actuator coupler may be configured to removably couple to the receiver coupler.

Any of the systems, devices, and methods disclosed herein may include any of the following features. The first coupler may be a magnet. The second coupler may be a magnet.

### BRIEF DESCRIPTION OF THE DRAWINGS

The accompanying drawings, which are incorporated in and constitute a part of this specification, illustrate aspects this disclosure and, together with the description, serve to explain the principles of the disclosure.

FIGS. 1A and 1B depict an exemplary medical device, according to aspects of this disclosure.

FIG. 1C depicts an alternative exemplary medical device, according to aspects of this disclosure.

FIGS. 2A-2D depict various views of a first aspect of a medical device, actuator assembly, and medical device system including the medical device and actuator assembly.

FIGS. 3A-3D depict various views of a second aspect of a medical device, actuator assembly, and medical device system including the medical device and actuator assembly.

FIGS. 4A-4C depict various views of a third aspect of a medical device, actuator assembly, and medical device system including the medical device and actuator assembly.

FIGS. 5A and 5B depict various views of a fourth aspect of a medical device, actuator assembly, and medical device system including the medical device and actuator assembly.

### DETAILED DESCRIPTION

An endoscope or other medical device (e.g., an endoscopic ultrasonography (“EUS”) scope, gastroscope, duodenoscope, ureteroscope, bronchoscope, cystoscope, colonoscope, etc.) may include an insertion portion and a handle. An operator may control various aspects of the medical device via one or more actuators, such as buttons, positioned on the handle of the medical device. For example, these actuators may control various features such as lighting, imaging (e.g., image capture), image processing, activation of image enhancement features, and the like, although such features are merely exemplary. Typically, single-use medical devices, including endoscopes or other similar devices, are disposed in their entirety, including the handle and actuators, after the medical device has been used for a medical procedure.

The disclosed medical devices and assemblies include reusable actuators, such as buttons. The actuators may be configured to control one or more aspects of the medical device, or a controller coupled to the medical device. The actuators may be elements of an assembly that is configured to be removably coupled to the medical device (e.g., to a handle of the medical device). In examples, the handle of the medical device may include a receiver for removably receiving and retaining an actuator assembly (e.g., a button assembly). The actuators may be wired or wirelessly coupled to one or more elements of a distal tip of the medical device or a controller of the medical device. Following a medical procedure, the actuator assembly may be uncoupled from the handle, and the handle may be disposed. The actuator assembly may be reused with another handle in a subsequent medical procedure.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values+/−10% of a stated value.

Throughout various figures, the arrows “P” and “D” depict proximal and distal directions. Any of the devices disclosed herein may include any of the following features, additionally or alternatively, in any combination. Accordingly, between different embodiments, like numbers will be used to refer to like features, with “100” added to each numeral.

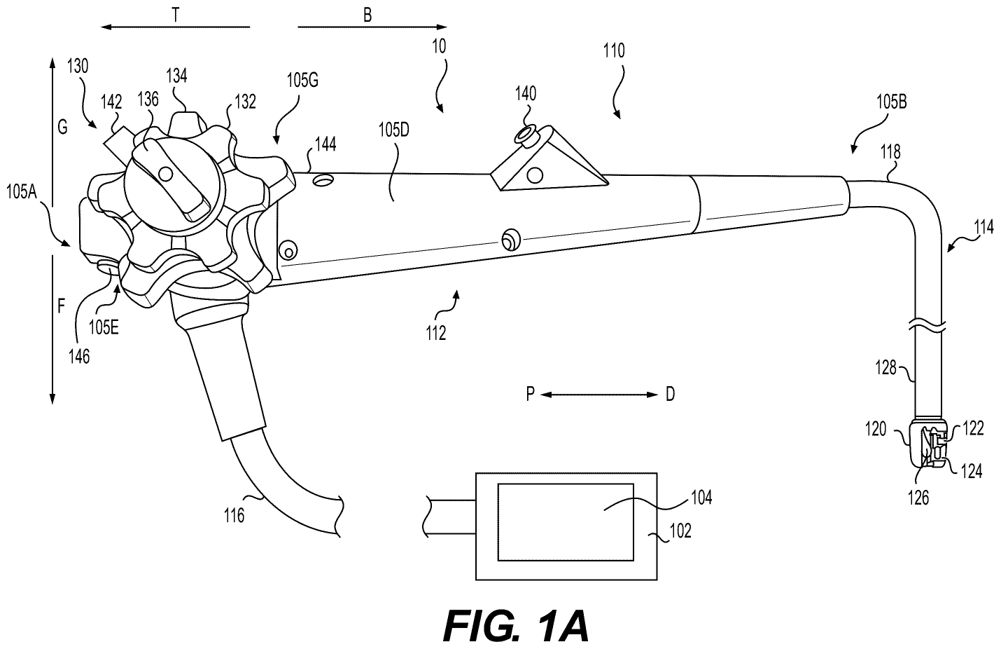

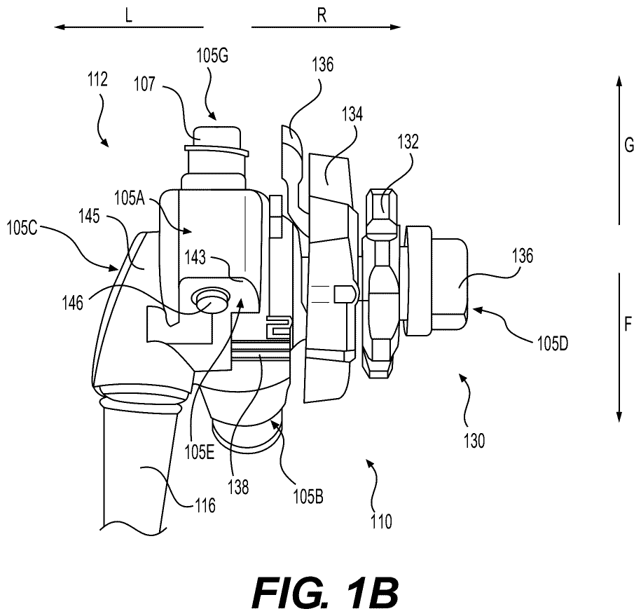

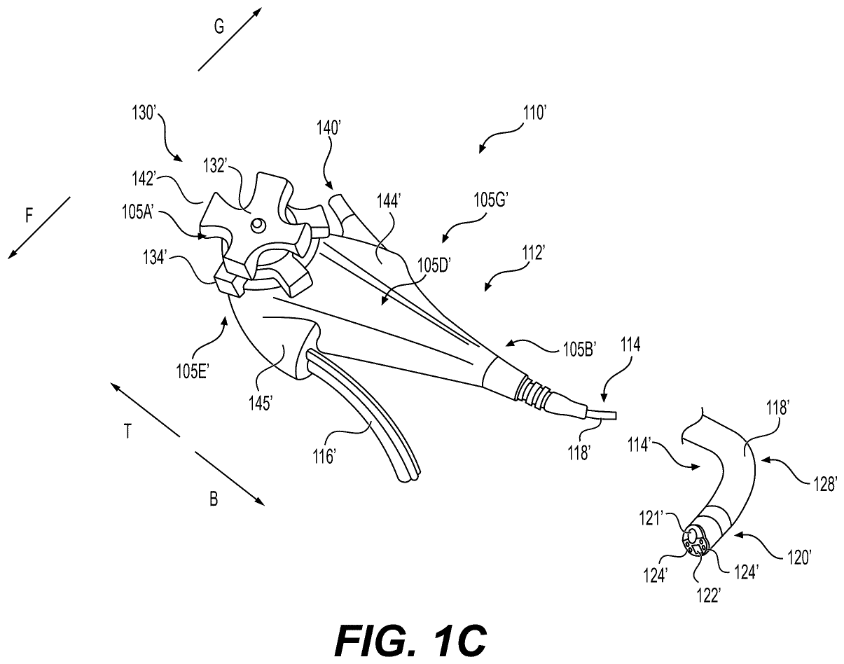

FIG. 1A depicts an exemplary medical system 10 including a medical device 110 and a controller 102. Medical device 110 may have a handle 112 and an insertion portion 114. FIG. 1B shows a proximal end of handle 112. Medical device 110 may also include an umbilicus 116 for purposes of connecting medical device 110 to sources of, for example, air, water, suction, power, etc., as well as to image processing and/or viewing equipment, such as controller 102. Although an endoscope may be referenced herein, it will be appreciated that the disclosure also encompasses duodenscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to an endoscope herein should be understood to encompass any of the above medical devices. Medical device 110 may be part of a system 10. System 10 may include medical device 110, a controller 102, and a user interface 104.

Insertion portion 114 may include a shaft 118, an articulation portion 128, and a distal tip 120. Distal tip 120 may include an imaging device 122 (e.g., a camera) and a lighting source 124 (e.g., an LED or an optical fiber). Distal tip 120 may be side-facing. That is, imaging device 122 and lighting source 124 may face radially outward, perpendicularly, approximately perpendicularly, or otherwise transverse to a longitudinal axis of shaft 118 and distal tip 120. Alternatively, imaging device 122 and lighting source 124 may face distally (be forward-facing). Additionally or alternatively, distal tip 120 may include one or more imaging devices 122 that face in more than one direction. For example, a first imaging device 122 may face radially outward, and a second imaging device 122 may face distally (e.g., approximately parallel to a longitudinal axis of distal tip 120/shaft 118).

Distal tip 120 may also include an elevator 126 for changing an orientation of a tool inserted in a working channel of medical device 110. Elevator 126 may alternatively be referred to as a swing stand, pivot stand, raising base, or any other suitable term. Elevator 126 may be pivotable, e.g., via one or more elevator wire(s) that extends from handle 112, through shaft 118 and articulation portion 128, to elevator 126.

In aspects, articulation portion 128 may be fixed (directly or indirectly) to a distal end of shaft 118 and a proximal end of distal tip 120. Articulation portion 128 may be comprised of, for example, one or more articulation joints. Shaft 118 and articulation portion 128 may include a variety of structures, which are known or may become known in the art. One or more articulation wires may extend from handle 112, through shaft 118, to a distal end of articulation portion 128. The articulation wire(s) may enable deflection of articulation portion 128 in one or more directions (e.g., up, down, left, right, etc.).

Referring still to FIGS. 1A and 1B, handle 112 may have one or more actuators/control mechanisms 130. Control mechanisms 130 may provide control of articulation portion 128 and/or may allow for provision of air, water, suction, etc. For example, handle 112 may include control knobs 132, 134 to enable deflection of articulation portion 128 in one or more directions. For example, one of knobs 132, 134 may provide left/right control of articulation portion 128, and the other of knobs 132, 134 may provide up/down control of articulation portion 128. Handle 112 may further include a first locking mechanism 136 and a second locking mechanism 142 (e.g., knobs or levers) for preventing steering and/or braking of articulation portion 128 in at least one of an up, down, left, or right direction. Handle 112 may include an elevator control lever 138 (see FIG. 1B). Elevator control lever 138 may raise and/or lower elevator 126, via connection between elevator control lever 138 and elevator wire(s) that extends from elevator control lever 138, through shaft 118, to elevator 126. One or more valve actuators 107 may be used to control suction, irrigation, and/or insufflation to distal tip 120. Handle 112 may include a button 146. Button 146 may be operatively coupled to one or more functional elements of medical device 110. For example, actuation of button 146 may cause imaging device 122 to capture an image.

Handle 112 may include a handle housing 144. Handle housing 144 may encase various internal elements of handle 112, such as control wires. Handle housing 144 may include various elements of handle 112, such as a port 140, for example. Port 140 of handle 112 may allow passage of a tool through port 140, into a working channel of the medical device 110, through shaft 118 and articulation portion 128, and to distal tip 120.

In some aspects of the disclosure, handle housing 144 may be configured to operatively accept one or more button assemblies (e.g., handle housing 144, or a portion thereof, may mate with one or more button assemblies). For example, handle housing 144 may be configured to removably accept one or more button assemblies in place of (or in addition to) button 146. An operator may grip handle housing 144 during a medical procedure with one hand, while the operator controls one or more actuators or buttons with another hand. In another example, an operator may grip handle housing 144 and control one or more actuators or buttons with the same hand.

Handle 112 may have various sides/surfaces, which are arranged for operability by an operator with one or more hands. When handle 112 is gripped by an operator, it may be held with a top side 105A (or proximal side) facing upward. A bottom side 105B (or distal side) may be opposite top side 105A. Shaft 118 may extend from bottom side 105B. The label “T” in FIG. 1A points toward the top direction, and the label “B” in FIG. 1 points toward the bottom direction. The top direction may be the same as the labeled proximal direction, and the bottom direction may be the same as the labeled distal direction.

A right side 105D of handle 112 may include control knobs 132 and 134 and locking mechanisms 136 and 142. A left side 105C may be a side that is opposite right side 105D. The label “R” in FIG. 1B shows a rightward direction and the label “L” in FIG. 1B shows a leftward direction. The right and left directions are substantially perpendicular to the top and bottom directions.

A front side 105E of handle 112 may face an operator (e.g., a user) while handle 112 is in use. Front side 105E may include button 146 and/or the various button assemblies and receiver assemblies described throughout this disclosure. Front side 105E on a surface 143 and may also include elevator control lever 138. Front side 105E and the actuators associated with front side 105E may be readily accessible by an operator's thumb during use of medical device 110 such that the various button assemblies and receiver assemblies described in this disclosure may be ergonomically accessed and controlled by an operator (e.g., an operator may actuate a button of a button assembly disposed on front side 105E). In an alternative, top side 105A may include the various button assemblies and receiver assemblies described throughout this disclosure. In another alternative, back side 105G may include button 146 and/or the various button assemblies and receiver assemblies described throughout this disclosure. A location of the button(s) and receiving assembly (or assemblies) may be chosen to provide ergonomic benefits so that a user may operate medical device 110 with less fatigue and/or with one hand.

A back side 105G (FIG. 1A) is opposite front side 105E and may include port 140 and valve actuators 107. The axis defining front and back is perpendicular to the axis defining left and right and is shown with arrows “F” (front) and “G” (back) in FIG. 1A. The axis defining front and back may extend into and out of the page of FIG. 1B.

Left side 105C of handle housing 144 may include a protruding portion 145. Umbilicus 116 may be coupled to protruding portion 145, on a front side of protruding portion 145, such that umbilicus 116 extends in an approximately front-ward direction from handle 112. It will be apparent to one of ordinary skill in the art that the various orientations described above may be applicable to any of the aspects of this disclosure.

In use, an operator may insert at least a portion of insertion portion 114 into a body lumen of a subject. Distal tip 120 may be navigated to a procedure site in the body lumen. Articulation portion 128 may be deflected to assist in accessing the procedure site within the subject. The operator may insert an accessory device (not shown) into port 140 and pass the accessory device through insertion portion 114 via a working channel to distal tip 120. The accessory device may exit the working channel at distal tip 120. The user may use elevator control lever 138 to raise elevator 126 and angle the accessory device toward a desired location (e.g., a papilla of the pancreatico-biliary tract). The user may use the accessory device to perform a medical procedure.

Controller 102 may be operatively and/or communicatively coupled to medical device 110. Controller 102 may be connected to medical device 110 via umbilicus 116 and/or various mechanisms of wireless communication. Controller 102 may have features of any controller or other type of capital equipment utilized with scope devices or other medical devices. For example, controller 102 may be a computer, tablet, or smartphone and the like including a user interface 104. User interface 104 may include various elements configured for user interaction (such as a touchscreen or mouse cursor) and displaying information. Controller 102 may include one or more processing units configured to process information (e.g., sensor data, imaging data, light data, etc.) received from medical device 110.

FIG. 1C shows an alternative medical device 110′ having a handle 112′ and a shaft 118′. Medical device 110′ may be used together with medical device 110 or separately from medical device 110. Medical device 110′ may have an insertion portion 114′ and a handle 112′.

Insertion portion 114′ may include a shaft 118′, an articulation portion 128′ (having any of the properties of articulation joint 128), and a distal tip 120′. Distal tip 120′ may include an imaging device 122′ (e.g., a camera) and lighting sources 124′ (e.g., LEDs or an optical fiber). Distal tip 120′ may be forward-facing. That is, imaging device 122′ and lighting sources 124′ may face distally, approximately parallel to a longitudinal axis of shaft 114′. Alternatively, imaging device 122′ and lighting sources 124′ may face distally (be forward-facing). Additionally or alternatively, distal tip 120′ may include one or more imaging devices 122′ that face in more than one direction. For example, a first imaging device 122′ may face radially outward, and a second imaging device 122′ may face distally (e.g., approximately parallel to a longitudinal axis of distal tip 120′/shaft 118′). Distal tip 120′ may also include a working channel distal opening 121.

Handle 112′ may have one or more actuators/control mechanisms 130. Control mechanisms 130′ may provide control of articulation portion 128′ and/or may allow for provision of air, water, suction, etc. For example, handle 112′ may include control knobs 132′, 134′ to enable deflection of articulation portion 128′ in one or more directions. For example, one of knobs 132′, 134′ may provide left/right control of articulation portion 128′, and the other of knobs 132′, 134′ may provide up/down control of articulation portion 128′. A locking mechanism (e.g., lever) 142′ may serve to lock a position of articulation portion 128′.

Handle 112 may include a handle housing 144′. Handle housing 144′ may encase various internal elements of handle 112′, such as control wires. Handle housing 144′ may include various elements of handle 112′, such as a port 140′, for example. Port 140′ of handle 112′ may allow passage of a tool through port 140′, into a working channel of the medical device 110′, through shaft 118′ and articulation portion 128′, and to distal tip 120′.

In some aspects of the disclosure, handle housing 144′ may be configured to operatively accept one or more button assemblies (e.g., handle housing 144′, or a portion thereof, may mate with one or more button assemblies). An operator may grip handle housing 144′ during a medical procedure with one hand, while the operator controls one or more actuators or buttons with another hand. Alternatively, handle 112′ may be coupled to a handle of another medical device, such as handle 112 or medical device 110.

Handle 112′ may have various sides/surfaces, which are arranged for operability by an operator with one or more hands. When handle 112′ is gripped by an operator, it may be held with a top side 105A′ (or proximal side) facing upward. A bottom side 105B′ (or distal side) may be opposite top side 105A′. Shaft 118′ may extend from bottom side 105B′. The label “T” in FIG. 1C points toward the top direction, and the label “B” in FIG. 1 points toward the bottom direction. The top direction may be the same as the labeled proximal direction, and the bottom direction may be the same as the labeled distal direction.

A right side 105D′ of handle 112′ may include control knobs 132′ and 134′ and locking mechanism 142′. A left side (not shown) may be a side that is opposite right side 105D′. The right and left directions are substantially perpendicular to the top and bottom directions.

A front side 105E′ of handle 112′ may face an operator (e.g., a user) while handle 112′ is in use. Front side 105E′ and actuators associated with front side 105E′ (such as those discussed above) may be readily accessible by an operator's thumb during use of medical device 110′ such that the various button assemblies and receiver assemblies described in this disclosure may be ergonomically accessed and controlled by an operator (e.g., an operator may actuate a button of a button assembly disposed on front side 105E′).

A back side 105G′ (FIG. 1C) is opposite front side 105E′ and may include port 140′. The axis defining front and back is perpendicular to the axis defining left and right and is shown with arrows “F” (front) and “G” (back) in FIG. 1A. The axis defining front and back may extend into and out of the page of FIG. 1B.

Front side 105E′ of handle housing 144′ may include a protruding portion 145′. Umbilicus 116′ may be coupled to protruding portion 145′, on a front side of protruding portion 145′, such that umbilicus 116′ extends distally/toward bottom surface 105B′. It will be apparent to one of ordinary skill in the art that the various orientations described above may be applicable to any of the aspects of this disclosure.

In use, distal tip 120′ of medical device 110′ may be inserted into port 140 of medical device 110. Shaft 118′ of medical device 110′ may be passed through a working channel of shaft 118 of medical device 110 until distal tip 120′ of medical device 110′ extends out of distal tip 120 of medical device 110. Alternatively, medical device 110′ may be used without medical device 110.

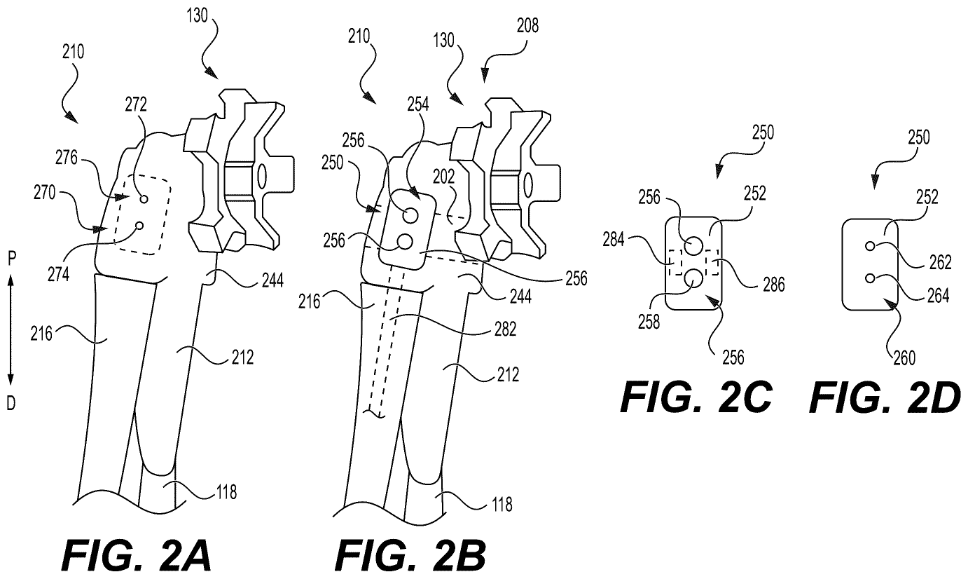

FIG. 2A shows a medical device 210 having a handle 212. FIG. 2B shows a medical system 208 including medical device 210 and an actuator or button assembly 250 removably attached to handle 212. FIG. 2C shows a first side 254 of button assembly 250. FIG. 2D shows a second side 260 of button assembly 250. Although the term “button assembly” 250 is used herein for convenience of reference, it will be appreciated that button assembly 250 may include any suitable type of actuator (e.g., levers, switches, toggles, knobs, joysticks, or sliders).

Medical device 210, as well as the other various systems, devices, and methods discussed throughout this disclosure, may allow an operator to attach and remove a button assembly, such as button assembly 250, from medical device 210. Medical device 210 may have any of the features of medical device 110, unless otherwise noted. In particular, handle 212 may have any of the properties and orientations of handle 112, described above. Where feasible throughout this disclosure, reference numbers ending in the same two digits (tens and ones digits) are used to identify similar structures. Medical device 210 may include a handle housing 244, which may encase various internal elements of handle 212, such as control wires, fluidics, steering components, a working channel, etc. Button assembly 250, described in further detail below, may be used on handle 112′ or in place of or in addition to button 146 of handle 112. Handle housing 244 may include a receiver assembly 270 for removably receiving button assembly 250, as described in further detail below.

Button assembly 250 includes a button housing 252. The term “button housing” is used to distinguish from handle housing 244, but, as discussed above, button assembly 250 may alternatively include actuators other than buttons. Button housing 252 may include a first side 254 and a second side 260. Button housing 252 is depicted with a rounded rectangular shape to correspond with receiver assembly 270 (e.g., button housing 252 and receiver assembly 270 may be correspondingly shaped), though this is only exemplary and other shapes of both button housing 252 and receiver assembly 270 are possible. Button housing 252 and receiver assembly 270 may have complementary shapes to facilitate removably coupling housing 252 to receiver assembly 270. Button housing 252 may have a substantially flat shape (e.g., a distance between first side 254 and second side 260 may be relatively small) so that button assembly 250 does not substantially protrude from a surface of handle housing 244. Button housing 252 may be sized and shaped so as to be received on an appropriate surface of handle housing 244 (e.g., such as a surface sized and shaped like surface 143 of handle housing 144).

Button housing 252 may be grasped by an operator when attaching or removing button assembly 250 from medical device 210. In some aspects, button housing 252 may include ergonomic features, such as raised and graspable protrusions on first side 254 to allow for improved manipulation by an operator.

Button assembly 250 may include a first side 254, shown in FIG. 2C. First side 254 may be understood as the side exposed to an operator (e.g., in a direction corresponding to direction F, toward front side 105E of FIGS. 1A and 1B and front side 105E′ of FIG. 1C) when button assembly 250 is attached to medical device 210. Button assembly 250 may include one or more buttons, such as button 256 and button 258. While two buttons are shown in FIG. 2C, any suitable number of buttons may be used with the systems, devices, and methods of the disclosure. As explained above, although the term “button” is used herein for ease of reference, it will be appreciated that buttons 256 and 258 may be alternative types of actuators.

Buttons 256 and 258 may each correspond with operation of one or more functions, such as lighting, imaging (e.g., image capture), image processing, activation of image enhancement features, etc. For example, button 256 may be used to capture an image with imaging device 122, while button 256 may be used to turn on or turn off a lighting source 124. However, the above functions are merely exemplary, and buttons 256 and 258 may control any aspect of medical device 210. In some aspects, combinations of buttons may be held by an operator to assign additional functionality to button assembly 250 without increasing the number of buttons. For example, an operator may actuate button 256 and button 258 simultaneously to activate an image enhancement feature, for example. In some aspects, button press sequences may be assigned to the one or more buttons of button assembly 250 to still further increase the number of functions accessible to an operator with a limited number of buttons. Buttons 256 and/or 258 may be programmable by a user to assign their functions or may be pre-programmed. In some aspects, the length of time (duration) of a button press may correspond with one or more functions (e.g., a one-second button press may correspond with a different function than a three-second button press).

Opposite the first side 254 of button assembly 250 is second side 260, shown in FIG. 2D. Second side 260 includes a first coupler 262 and a second coupler 264. In some examples, a position of first coupler 262 may correspond approximately to a position of button 256, and a position of second coupler 264 may, correspond approximately to a position of button 258. However, such an arrangement is merely exemplary. Couplers 262 and 264 may include magnets configured to magnetically attach to corresponding magnets of receiver assembly 270, as discussed below. Second side 260 may abut and face medical device 210 (e.g., handle 212) while button assembly 250 is connected to medical device 210 (e.g., to handle 212). For example, second side 260 may be coupled to front side 105E of handle 112 of FIGS. 1A and 1B or to front side 105E′ of handle 112 of FIG. 1C. Such a position may be advantageous to allow a thumb of an operator to actuate buttons 256, 258. Alternatively, second side 260 may be coupled to another portion of handle 212.

Second side 260 may also be described as facing the back side. While two couplers 262 and 264 are shown in FIG. 2C, it will be understood that other numbers of couplers are possible. In some aspects, the number of couplers of button assembly 250 may not be equal to the number of buttons. For example, a single large magnet may act as a coupler for several buttons, as discussed below, or a number of couplers may exceed a number of buttons.

Returning to medical device 210 and FIG. 2A, handle housing 244 of handle 212 may include a receiver assembly 270. In some aspects, receiver assembly 270 may be coupled to a portion of handle 212 corresponding to front side 105E or 105E′. Receiver assembly 270 may include a first coupler 272 and a second coupler 274. First coupler 272 and second coupler 274 may each be a magnet (e.g., a neodymium magnet); however, other types of couplers (e.g., snap-fits, friction fit couplers, hook and loop) are also within the scope of the disclosure. Receiver assembly 270 may mate with button assembly 250 via the interactions of first coupler 272 with coupler 262, and coupler 274 with second coupler 264. First coupler 272 and second coupler 274 may be magnets with a suitable (e.g., opposite) polarity of the corresponding magnets of couplers 262 and 264 of button assembly 250.

Receiver assembly 270 may optionally include visual markings along its perimeter to inform an operator where to place button assembly 250. First coupler 272 and second coupler 274 may optionally include markings, colors, or indications corresponding to like markings, colors, or indications on first coupler 262 and second coupler 264. These visual references may enhance an operator's experience in mating button assembly 250 with receiver assembly 270. In some aspects, receiver assembly 270 may include a recess 276 corresponding to the shape of button assembly 250. Recess 276 may be of a suitable depth to prevent or inhibit unintended lateral movement of button assembly 250 when an operator actuates one or more buttons (e.g., buttons 256 and 258) and/or protrusion of button assembly 250 beyond a surface of handle housing 244.

In use, an operator may grasp and align button assembly 250 with receiver assembly 270. Specifically, the operator may place second side 260 into close contact with receiver assembly 270 (e.g., with first side 254 facing the operator). The operator may be guided by one or more visual indicators on couplers 262, 264, 262, and 274 and/or via recess 276. Magnetic force (or another type of coupling mechanism) may then join coupler 262 to coupler 272, and second coupler 264 to coupler 274, respectively (shown in FIG. 2B). In some aspects of the disclosure, button assembly 250 or a portion thereof may be substantially flush with or parallel to receiver assembly 270 and/or handle housing 244. The attachment of the couplers may attach button assembly 250 to receiver assembly 270 such that an operator may manipulate medical device 210 without button assembly 250 detaching in an unwanted manner.

In an alternative, couplers 262, 264, 272, and/or 274 and/or receiver assembly 270 may be omitted, and button assembly 250 may be coupled to handle 212 with an optional strap 202 (FIG. 2B). Strap 202 may be composed of an elastomer, such as silicone or rubber. A position of strap 202 in FIG. 2B is shown merely for illustration, and strap 202 may have any alternative position. For example, a position of strap 202 may be chosen so that there is no interference with actuators of handle 212. In such aspects, an operator may place button assembly 250 onto handle 212, with second side 260 facing housing 244. Strap 202 and/or second side 260 may abut housing 244. In some aspects, strap 202 may include a receiver having any structure suitable to retain button assembly 250. In alternatives, an operator may place strap 202 across first side 254 such that button assembly 250 remains removably fixed to receiver assembly 250.

Button assembly 250 may be operatively connected to controller 102 via one or more of various mechanisms. For example, a wired connection may operatively connect button assembly 250 and controller 102. For example, one or more optional wire(s) 282 (FIG. 2B) may be electrically coupled to elements of button assembly 250 and may extend from button assembly 250 to a controller and/or to a connector of an umbilicus 216 of medical device 210. Wire(s) 282 may be directly coupled to button assembly 250 and/or may emanate from button assembly 250. In alternatives, wire(s) 282 may emanate from receiver 270, and couplers 262 and/or 264 and 272 and/or 274 may form electrical contacts to couple button assembly 250 to wire(s) 282. In aspects, the wired connection via wire(s) 282 may provide electrical power to button assembly 250.

In aspects, a button assembly 250 having a wired connection via wire(s) 282 between button assembly 250 and controller 102 may omit a microcontroller and/or a microprocessor and/or a processor (or other electrical components). In such examples, actuation of the one or more buttons 256, 258 may generate a signal that may be transmitted via the wired connection directly to controller 102 for further processing. In some aspects with two or more wires, a first wire may be used only for signaling, and a second wire or a battery may provide power to the microcontroller or microprocessor.

In another aspect, second side 260 may include one or more electrical connectors (e.g., contacts) (e.g., couplers 262 and/or 264 or other contacts) configured to electrically and/or communicatively couple with one or more electrical connectors (e.g., contacts) disposed on receiver assembly 270 (e.g., couplers 272 and/or 274 or other contacts). Power may be routed from controller 102, through umbilicus 216, to medical device 210 (e.g., via handle 212), through the one or more electrical contacts of receiver assembly 270, and to the one or more electrical contacts of button assembly 250. Examples of electrical contacts include electrical connectors, contact pads, pogo pins, spring-loaded pins, electrical couplers, flush electrical contacts, and the like. In some aspects, the receiver assembly 270 electrical connector(s) may be electrically connected to umbilicus 216.

In another aspect, a wireless connection may operatively connect button assembly 250 with controller 102 (e.g., via Bluetooth, Wi-Fi, and/or NFC). In such aspects, button assembly 250 may include one or more batteries 284 and one or more electronic elements 286, One or more electronic elements 286 may include one or more wires, mechanisms for signaling (e.g., transceivers, transmitters, or receivers), a microcontroller, and/or a microprocessor or other processor suitable for interpreting or detecting inputs (e.g., actuation of one or more buttons 256, 258) and/or encoding outputs or signal or indications (e.g., information corresponding to actuation of the one or more buttons) to the transceiver for transmission. The signaling components, processor(s)/controller(s), and batterie(s) may be electrically connected to one another. The batter(ies) 284 and electronic element(s) 286 may be disposed within button housing 252 in at least some aspects.

In some examples, button assembly 250 may include one or more electrical components (e.g., electronic element(s) 286 and/or batter(ies) 284). Button assemblies 250 having such electrical components may communicate using wired or wireless techniques, such as those discussed above. In an example, depressing or otherwise actuating one or more of buttons 256, 258 may cause a signal that a microprocessor (e.g., electronic element 286) of button assembly 250 may recognize, interpret, or detect. A transmitter or transceiver (e.g., another electronic element 286) of button assembly 250 may transmit a signal (e.g., a Wi-Fi or other wireless signal) to a receiver within controller 102 that is indicative of the actuation. Controller 102 may then send a signal to medical device 210 via umbilicus 216 in order to activate a desired function.

Alternatively or additionally, button assembly 250 may be in wired and/or wireless communication with elements of distal tip 120 directly, with or without communication with controller 102. Such aspects may include any of the features (e.g., electrical contacts, wires, batteries, electronic elements) discussed above. In examples, button assembly 250 may be communicatively linked with controller 102 and/or distal tip 120 through a handshake or other pairing procedure (e.g., a Bluetooth, Wi-Fi, and/or NFC pairing procedure).

In wireless aspects of the disclosure, batteries of button assembly 250 may be rechargeable. For example, the batteries may be recharged via a wired connection, such as a USB connection (e.g., via a charging port on button assembly 250). Alternatively or additionally, charging of the batteries of button assembly 250 may be performed wirelessly by wireless charging mechanisms such as electromagnetic inductive (e.g., non-radiative) charging, loosely coupled (e.g., radiative electromagnetic resonant) charging, and/or uncoupled radio frequency (RF) charging. The aforementioned wireless charging mechanisms may be built into handle 112, which may allow for charging while medical device 110 is in use by an operator.

Buttons 256 and 258 may be actuated by an operator to control one or more functions at distal tip 120, such as image capture with imaging device 122 or adjusting of lighting source 124. For example, an operator may actuate button 256 to capture an image with imaging device 122. In another example, an operator may press and hold button 256 to capture a video with imaging device 122. In another example, actuation of button 256 may correspond with a captured image being displayed on user interface 104 of controller 102. At the end of the medical procedure, an operator may grasp button assembly 250 and pull with sufficient force to separate couplers 262, 264, 272, and 274, thereby removing button assembly 250 from medical device 210. Button assembly 250 may then be suitably sterilized and/or cleaned and used for a future medical procedure with a different medical device 210, thereby avoiding disposal of button assembly 250. Button assembly 250 may also be used to optionally supplement medical devices (e.g., medical devices 110, 110′, or 210) for use in certain medical procedures that require one or more buttons. In some aspects, button-assembly 250 may be single-use.

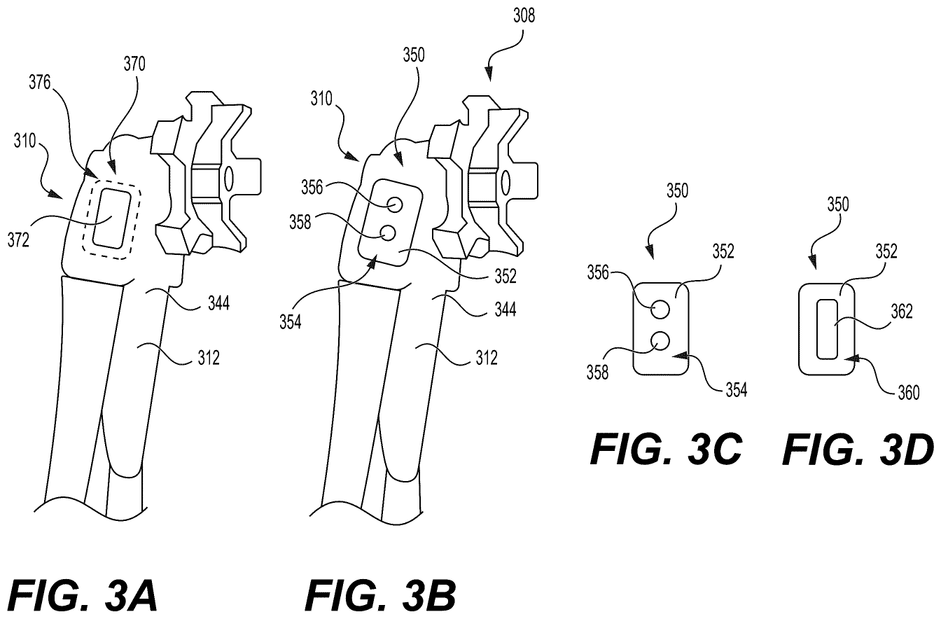

FIG. 3A shows a medical device 310 having a handle 312. FIG. 3B shows a medical system 308 including medical device 310 and an actuator or button assembly 350 removably attached to handle 312. FIG. 3C shows a first side 354 of button assembly 350. FIG. 3D shows a second side 360 of button assembly 350.

Medical system 308 may have any of the features of medical system 208, above, unless otherwise specified below. Medical device 310 may have any of the features of medical device 110 and medical device 210, unless otherwise noted. Medical device 310 may include a handle housing 344, which may encase various internal elements of handle 312, such as control wires, fluidics, steering components, a working channel, etc. Button assembly 350, described in further detail below, may be used on handle 112′ or in place of or in addition to button 146 of handle 112. Button assembly 350 may have any of the features of button assembly 250, unless otherwise specified. Handle housing 344 may include a receiver assembly 370 for removably receiving button assembly 350, as described in further detail below. Receiver assembly 370 may have any feature of receiver assembly 270, unless otherwise noted.

Button assembly 350 includes a button housing 352. The term “button housing” is used to distinguish from handle housing 344, but, as discussed above, button assembly 350 may alternatively include actuators other than buttons. Button housing 352 may include a first side 354 and a second side 360. Button housing 352 is depicted with a rounded rectangular shape to correspond with receiver assembly 370, though this is only exemplary and other shapes of both button housing 352 and receiver assembly 370 are possible. Button housing 352 and receiver assembly 370 may have complementary shapes to facilitate removably coupling button housing 352 to receiver assembly 370. Button housing 352 may have a substantially flat shape (e.g., a distance between first side 354 and second side 360 may be relatively small) so that button assembly 350 does not substantially protrude from a surface of handle housing 344. Button housing 352 may be grasped by an operator when attaching or removing button assembly 350 from medical device 310. In some aspects, button housing 352 may include ergonomic features, such as raised and graspable protrusions on first side 354 to allow for improved manipulation by an operator.

Button assembly 350 may include a first side 354, shown in FIG. 3C. First side 354 may be understood as the side exposed to an operator when button assembly 350 is attached to medical device 310. Button assembly 350 may include one or more buttons, such as button 356 and button 358. While two buttons are shown in FIG. 3C, any suitable number of buttons may be used with the systems, devices, and methods of the disclosure. As explained above, although the term “button” is used herein for ease of reference, it will be appreciated that buttons 356 and 358 may be alternative types of actuators.

Opposite the first side 354 of button assembly 350 is second side 360, shown in FIG. 3D. Second side 360 includes a coupler 362, corresponding approximately to a position (e.g., area) of both button 256 and button 258. Coupler 362 may include a larger, single magnet configured to magnetically attach to a corresponding magnet of receiver assembly 370, as discussed below. Compared to the couplers 262 and 264 discussed above with respect to FIGS. 2A-2D, coupler 362 may be larger. Coupler 362 may occupy a portion of second side 360 that corresponds to portions of first side 354 including both of buttons 256 and 257. Coupler 362 is shown with a rounded rectangular shape in FIG. 3D, though this is only exemplary and other shapes are possible. Second side 360 may abut and face medical device 310 (e.g., handle 312) while button assembly 350 is connected to medical device 310 (e.g., to handle 312).

Returning to medical device 310 and FIG. 3A, handle housing 344 of handle 312 may include a receiver assembly 370. Receiver assembly 370 may include a coupler 372. Coupler 372 may be a magnet (e.g., a neodymium magnet); however, other types of couplers (e.g., snaps, friction fit couplers, hook and loop) are also within the scope of the disclosure. Although only one coupler 372 is shown, it will be appreciated that receiver assembly 370 may have a plurality of couplers 372. In some examples, coupler 372 may have a shape that complements a shape of coupler 362. Receiver assembly 370 may mate with button assembly 350 via the interactions of coupler 362 and coupler 372. Coupler 362 may be a magnet with a suitable (e.g., opposite) polarity of the corresponding magnet of coupler 372 of button assembly 350 (e.g., the magnet of coupler 362 may interact with the magnet of coupler 372).

Receiver assembly 370 may optionally include visual markings along its perimeter to inform an operator where to place button assembly 350. Coupler 372 may optionally include markings, colors, or indications corresponding to like markings, colors, or indications on coupler 362. In some aspects, receiver assembly 370 may include a recess 376 corresponding to the shape of button assembly 350. Recess 376 may be of a suitable depth to prevent unintended lateral movement of button assembly 350 when an operator actuates one or more buttons (e.g., buttons 356 and 358) and/or protrusion of button assembly 350 beyond a surface of handle housing 344. It will be understood by one of ordinary skill in the art that any of the above mentioned uses, functionality, and operational techniques described previously with respect to medical system 208, medical device 210, and button assembly 250 may be applicable to medical system 308, medical device 310, and button assembly 350, and any subcomponent thereof.

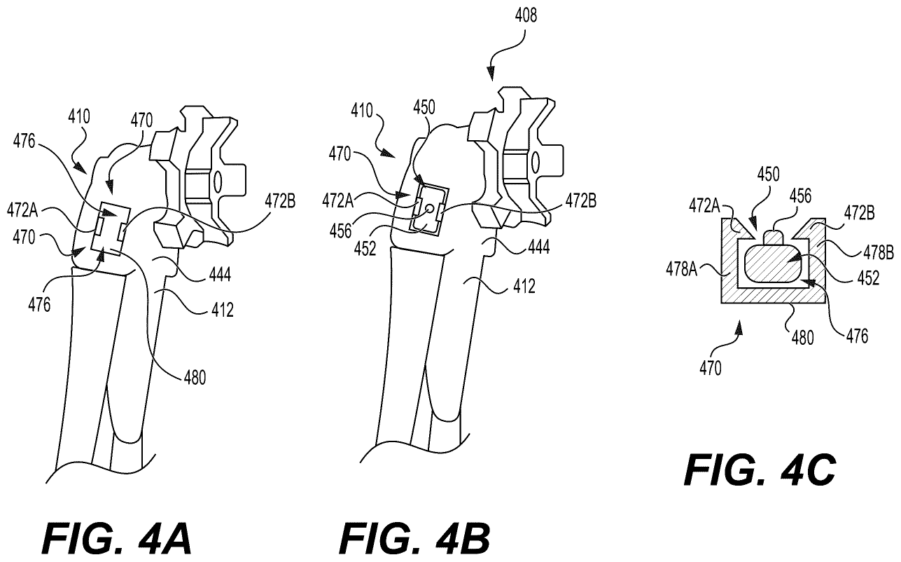

FIG. 4A shows a medical device 410 having a handle 412. FIG. 4B shows a medical system 408 including medical device 410 and an actuator or button assembly 450 removably attached to handle 412. FIG. 4C shows a cross-sectional view of button assembly 450 mated to a receiver assembly 470 of handle 412.

Medical system may have any of the features of medical systems 208, 308, unless otherwise noted. For example, medical device 410 may have any of the features of medical devices 110, 210, and 310, unless otherwise noted Medical device 410 may include a handle housing 444, which may encase various internal elements of handle 412, such as control wires, fluidics, steering components, a working channel, etc. Button assembly 450, described in further detail below, may be used on handle 112′ or in place of or in addition to button 146 of handle 112. Handle housing 444 may include receiver assembly 470 for removably receiving button assembly 450, as described in further detail below. Receiver assembly 470 may have any feature of receiver assemblies 270 and 370, unless otherwise noted.

Button assembly 450 includes a button housing 452. The term “button housing” is used to distinguish from handle housing 444, but, as discussed above, button assembly 450 may alternatively include actuators other than buttons. Button housing 452 may be grasped by an operator when attaching or removing button assembly 450 from medical device 410. Button assembly 450 may include one or more buttons, such as button 456. While only one button is shown in FIG. 4C, any suitable number of buttons may be used with the systems, devices, and methods of the disclosure. As explained above, although the term “button” is used herein for ease of reference, it will be appreciated that button 456 may be alternative types of actuators. Button 456 should be understood to be exposed to an operator when button assembly 450 is attached to medical device 410.

Returning to medical device 410 and FIG. 4C, handle housing 444 of handle 412 may include a receiver assembly 470. Receiver assembly 470 may be a snap fit mechanism configured to removably couple to button assembly 450. Alternatively, button assembly 450 may slide into receiver assembly 470. Receiver assembly 470 may include a coupler arm 472A and a coupler arm 472B. Coupler arms 472A and 472B may each include a protrusion configured to prevent inadvertent movement (e.g., removal) of button assembly 450 from receiver assembly 470. For example, button assembly 450 may not fall out of receiver assembly 470 if held upside down (e.g., with button assembly 450 facing the ground) due the snap locking of coupler arms 472A and 472B with button assembly 450.

Coupler arm 472A may be joined (e.g., being integrally formed with) to a side wall 478A, and coupler arm 472B may be joined to a side wall 478B. Side walls 478A and 478 may be joined to an inner wall 480. Side walls 478A and 478B are shown joining inner wall 480 at an approximately 90 degree angle, though this is only exemplary and other angles are possible. Coupler arms 472A and 472B, side walls 478A and 478B, and inner wall 480 may define a cavity or recess 476. Recess 476 may be an approximately rectilinear volume configured to accept button assembly 450. Recess 476 may be of an approximate volume and depth such that button assembly 450 may fit within recess 476. It will be understood by one of ordinary skill in the art that the depicted shapes and proportions of button assembly 450 and receiver assembly 470 (e.g., as shown in FIG. 4C) may be modified without departing from the scope of the disclosure.

A user may grasp button assembly 450 and push (e.g., apply force to) button assembly 450 approximately perpendicularly to the plane defined by inner wall 480. Button assembly 450 may be pushed against coupler arm 472A and coupler arm 472B, such that the coupler arms 472A and 472B and side walls 478A and 478B may elastically deflect to accept button assembly 450 into recess 476. Coupler arms 472A and 472B and side walls 478A and 478B may then then elastically deflect back to the “default” or “rest” state depicted in FIG. 4C. In alternatives, button assembly 450 may be slid into recess 476. When button assembly 450 is received within recess 476, coupler arms 472A and 472B may extend at least partially along a top/outer surface of button assembly 450 (a surface on which button 456 is disposed).

With button assembly 450 at least partially surrounded by inner wall 480, side walls 478A and 478B, and coupler arms 472A and 472B, button assembly 450 may be removably coupled to medical device 410. Following a medical procedure, an operator may apply force to coupler arms 472A and 472B and side walls 478A and 478B to elastically deflect these elements. The operator may then grasp and remove button assembly 450 from receiver assembly 470. Alternatively, an operator may slide button assembly 450 out of receiver assembly 470. In some examples, button assembly 450 and/or receiver assembly 470 (e.g., inner wall 480) may include one or more magnets having any of the properties of the magnets discussed above.

In an alternative, receiver assembly 470 may be modified such that mating with button assembly 450 is achieved by sliding button assembly laterally (e.g., along a plane approximately parallel to inner wall 480. In these alternatives, an opening may be provided above button 456 such that a user may actuate button 456. Button assembly 450 (and any of the other button assemblies described in this disclosure) may have any of the communication mechanisms/arrangements and/or electronic components described above).

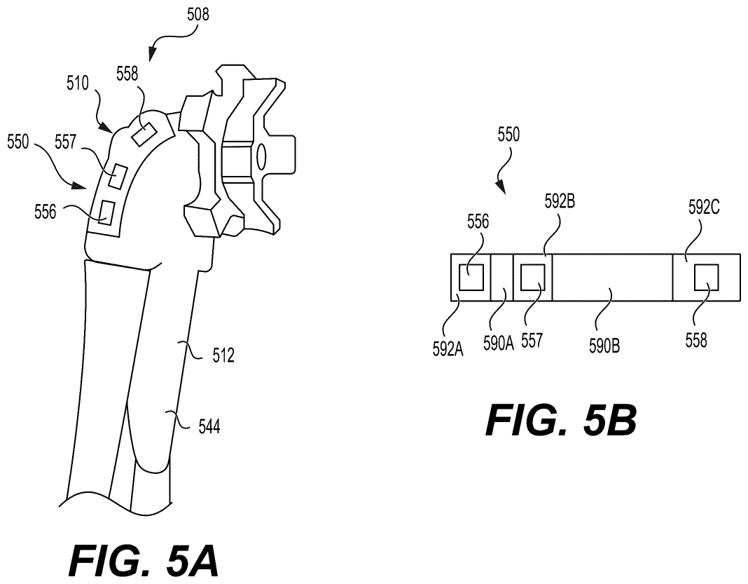

FIG. 5A shows a medical system 508, including a button assembly 550 and a medical device 510 having a handle 512. FIG. 5B shows a detailed view of button assembly 550. FIG. 5A may depict a flexible button assembly 550 configured to adjust and conform to a shape of handle 512. Flexible button assembly 550 may also be a rigid-flexible or semi-rigid button assembly. Although button assembly 550 is described as being flexible, it will be appreciated that any of the aspects above may also include flexible elements.

Medical system 508 may have any feature of medical systems 208, 308, 408, unless otherwise specified. For example, medical device 510 may have any of the features of medical devices 110, 210, 310, and 410, unless otherwise noted. Medical device 510 may include a handle housing 544, which may encase various internal elements of handle 512, such as control wires, fluidics, steering components, a working channel, etc. Button assembly 550, described in further detail below, may be used on handle 112′ or in place of or in addition to button 146 of handle 112. Handle housing 544 may include a receiver assembly for removably receiving button assembly 450, as described in further detail below. The receiver assembly may have any feature of receiver assemblies 270, 370, and 470, unless otherwise noted.

Button assembly 550 may include one or more flexible sections connected to one or more inflexible sections. Flexible sections 590A and 590B may be formed of rubber, silicone, polypropylene, ABS, polyethylene, polyimide (e.g., a flexible or rigid-flex circuit board material), or other flexible suitable materials. Button assembly 550 may include an alternating arrangement of flexible and inflexible (e.g., rigid) sections such that each flexible section is connected to at least one inflexible section and vice-versa. It the example shown in FIG. 5B, two flexible sections 590A, 590B and three inflexible sections 592A, 592B,592C are shown. It will be apparent to one of ordinary skill in the art that various other quantities and arrangements of flexible and inflexible sections are possible without departing from the scope of this disclosure.

One or more buttons may be joined to the one or more inflexible or rigid sections 592A, 592B, 592C. For example, a button 556 a may be joined to inflexible section 592A, a button 557 may be joined to inflexible section 592B, and a button 558 may be joined to inflexible section 592C. As explained above, although the term “button” is used herein for ease of reference, it will be appreciated that buttons 556, 557, and 558 may be alternative types of actuators. Buttons 556, 557, and 558 should be understood to be exposed to an operator when button assembly 550 is attached to medical device 510.

An operator may attach button assembly 550 to medical device 510 by various mechanisms, including any previously described in the disclosure. For example, the side of button assembly 550 facing medical device 510 may include an adhesive that removably joins button assembly 550 to medical device 510. In another example, the side of button assembly 550 facing medical device 510 may include one or more magnets configured to mate with one or magnets disposed on medical device 510, in a substantially similar manner as to those discussed above with respect to FIGS. 2 and 3. Other couplers (e.g., snaps, friction fit couplers, hook and loop, double-sided or other adhesive, such as a sticker) may also be used with medical system 508. In further examples, one or more recesses of medical device 510 may define surfaces similar to those described above with respect to FIGS. 4A-4C.

Flexible sections 590A and 590B may allow button assembly 550 to bend with (e.g., conform to) various curvatures of medical device 510 (e.g., handle 512). Flexible sections paired with a wireless configuration of button assembly 550 (e.g., including batteries, transceivers, and/or microcontrollers) may be suitable for retrofitting existing medical devices. For example, a user may attach button assembly 550 to a medical device handle to add additional functionalities to the medical device handle (e.g., lighting, image capture, image processing, activation of image enhancement features, and the like). It will be understood by one of ordinary skill in the art that any of the above-mentioned uses, functionality, and operational techniques described previously with respect to medical systems 208, 308, and 408 may be applicable to medical system 508 and any subcomponent thereof.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. A medical system comprising:
an actuator assembly comprising:
an actuator housing including a first side and a second side opposite the first side;
an electrical component;
an actuator disposed on the first side, exposed to a user of the medical system, and operably coupled to the electrical component; and
an actuator coupler proximate to the second side; and
a medical device comprising:
a handle including a handle housing;
an umbilicus for electrically connecting the handle to an electrical controller; and
a receiver assembly disposed on the handle housing, the receiver assembly including a receiver coupler,
wherein the actuator coupler is configured to removably couple to the receiver coupler.

2. The medical system of claim 1, wherein the electrical component comprises a battery, a transmitter or transceiver, and a processor, wherein the battery, the transceiver, and the processor are electrically connected.

3. The medical system of claim 2, wherein the processor is configured to detect actuation of the actuator, wherein the transmitter or transceiver is configured to transmit a signal indicating the actuation to the electrical controller.

4. The medical system of claim 1, wherein the receiver assembly is disposed on a front side of the handle housing and wherein the umbilicus is coupled to a protrusion extending from a left side of the handle housing or a front side of the handle housing.

5. The medical system of claim 4, further comprising:
a control knob; and
a locking mechanism operably coupled to the control knob,
wherein the control knob and the locking mechanism are disposed on a right side of the handle housing.

6. The medical system of claim 1, wherein the actuator coupler comprises an actuator magnet, wherein the receiver coupler comprises a receiver magnet.

7. The medical system of claim 1, receiver coupler comprises:
a first coupler arm and a second coupler arm;
a first side wall joined to the first coupler arm, and a second side wall joined to the second coupler arm; and
an inner wall joined to the first side wall and the second side wall.

8. The medical system of claim 7, wherein the first coupler arm and the second coupler arm are configured to snap-fit onto the actuator assembly.

9. The medical system of claim 1, wherein the actuator assembly comprises a flexible portion and a rigid portion, and wherein the rigid portion comprises the actuator.

10. The medical system of claim 3, wherein the electrical component further comprises an actuator electrical connector, wherein receiver assembly further comprises a receiver electrical connector, wherein actuation of the actuator corresponds with transmission of a signal from the actuator electrical connector to the receiver electrical connector.

11. The medical system of claim 10, wherein the receiver electrical connector is electrically connected to the umbilicus.

12. The medical system of claim 3, wherein the signal is transmitted wirelessly.

13. The medical system of claim 1, wherein the actuator assembly and the receiver assembly are correspondingly shaped, wherein the receiver assembly includes a recess, the recess configured to inhibit lateral movement of the actuator housing relative to the receiver assembly.

14. The medical system of claim 13, wherein the receiver coupler comprises a strap.

15. The medical system of claim 3, wherein the electrical controller is configured to control, based on the signal, at least one of an image capture function or a light control function of the medical device.

16. A medical system comprising:
an actuator assembly comprising:
a first side having an actuator;
a second side having a first magnet; and
an electrical component; and
a medical device handle including a receiver, wherein the receiver includes a second magnet configured to interact with the first magnet in order to removably couple the actuator assembly to the receiver.

17. The medical system of claim 16, wherein the electrical component comprises a battery, a transmitter or transceiver, and a processor, wherein the battery, the transceiver, and the processor are electrically connected.

18. The medical system of claim 17, wherein the transmitter or transceiver is configured to transmit a signal to an electrical controller indicating that the actuator has been actuated, and wherein the electrical controller is configured to control, based on the indication, at least one of an image capture function or a light control function of a distal tip of a shaft to the medical device handle.

19. A medical system comprising:
an actuator assembly comprising:
a transmitter or transceiver;
an actuator operably coupled to transmitter or transceiver; and
a first coupler; and
a medical device comprising:
a handle;
an umbilicus for electrically connecting the handle to an electrical controller; and
a receiver assembly disposed on the handle, the receiver assembly including a second coupler and a recess for removably receiving the actuator assembly,
wherein the actuator coupler is configured to removably couple to the receiver coupler.

20. The medical system of claim 19, wherein the first coupler is a magnet, and wherein the second coupler is a magnet.
